(19) United States
(12) Patent Application Publication
(10) Pub. No.: US 2024/0262858 A1
(43) Pub. Date: Aug. 8, 2024

(54) SCALABLE PRODUCTION OF POLYRIBONUCLEOTIDES OF CONTROLLED SIZE

(71) Applicant: Oncovir, inc., Stenvenson, MD (US)

(72) Inventors: Peter C. Michels, Albany, NY (US); Seth C. Pearson, Franklin, OH (US)

(73) Assignee: Oncovir, Inc., Stevenson, MD (US)

(21) Appl. No.: 17/803,950

(22) PCT Filed: Aug. 3, 2021
(86) PCT No.: PCT/US2021/010032

§ 371 Date: Feb. 2, 2023

Related U.S. Application Data
(60) Provisional application No. 63/103,487, filed on Aug. 5, 2020.

(51) Int. Cl. C07H 21/02; C12N 11/087

(52) U.S. Cl. CPC C07H 21/02; C12N 11/087

(57) ABSTRACT

A scalable process for production of polyribonucleotides of controlled molecular weight range through variation of processing time and input concentrations. Key elements include a method for immobilization of polynucleotide phosphorylase which has been covalently attached to an amino-functionalized solid support via a glutaraldehyde linkage; a method of repeatedly reacting inosine diphosphate or cytidine diphosphate monomer s with immobilized polynucleotide phosphorylase to produce polyribonucleotide chains; control of the chain length of Poly(I) and Poly (C) by varying cofactor concentration and the length of reaction time; a method for controlled and efficient large-scale manufacture of a specific, determined range of molecular weight poly I and poly C homopolymer chains.

### FIELD AND BACKGROUND OF THE INVENTION

### Field of the Invention

The present invention relates in general to production of polyribonucleotides, and more specifically to biocatalytic production of polyribonucleotides of controlled molecular weight range using immobilized polynucleotide phosphorylase.

### Background Information

The invention described and claimed herein comprises a method for repeatedly producing polyribonucleotides of a desired molecular weight by contacting an aqueous solution of nucleoside diphosphates with immobilized polynucleotide phosphorylase (PNPase).

PNPase catalyzes the synthesis of long polynucleotides from monomeric nucleoside diphosphates via introduction of a 3′,5′-phosphodiester bond. The same enzyme also catalyzes the reverse reaction, in which nucleoside diphosphates are removed by processive phosphorolysis of the polynucleotide. A divalent metal cation is required for catalysis, most frequently magnesium or manganese.

PNPase has been identified in and isolated (to varying degrees) from mammals, plants, and various bacteria including M. luteus, E. coli, A. agilis, A. vinelandii, V. costicola, S. antibioticus, B. stearothermophylus, T. thermophylus, C. perfringens, and various Achromobacter species (Creighton, 1999; Yamauchi et al, 1986; De Lassauniere et al, 1990; Soreq et al, 1977; Singer et al, 1960; Eckstein and Gindl, 1969; Rokugawa et al, 1988). The enzyme has also been overexpressed and isolated from recombinant E. coli (Marumo et al, 1993) and is currently commercially available as a purified recombinant product (Nipro, Japan).

The use of soluble PNPase to produce polyribonucleotides has been described extensively in the literature as well as in French Patent 2,114,198 (Pabst), German Patent 2,365,894 (Rokugawa), and U.S. Pat. No. 4,927,755 (De Lassauniere). Each of these methods require extraction, and concurrent destruction, of the PNPase biocatalyst during isolation of the polyribonucleotide product. As an alternative to the soluble enzyme process, immobilization of PNPase on a solid support has been considered. Immobilization confers several benefits to the process-notably, simplified separation of polynucleotide products from biocatalyst post-reaction in a non-destructive manner and therefore, possibility of reuse of the biocatalyst.

Immobilization of PNPase on a solid support has been described in French Patent 2,252,350 (Choay) which describes a Sepharose support and cyanogen bromide linkage, European Patent 0,346,865 (Moran) which describes various supports (acrylic, sepharose, PVA) and epoxy linkage, and European Patent 0,368,808 which describes a chitosan support and both physical adsorption and crosslinkages. However, these methods have certain disadvantages for large scale, economical production of a drug product for human use. These are described below on pages 17-19 in the Description of the Preferred Embodiment.

A specific polyribonucleotide of interest is a duplex referred to as “poly-IC”, double-stranded RNA (dsRNA), which can be formed by combining Polyinosinic acid (poly-I) and poly-cytidylic acid (poly-C). This duplex can be further stabilized with poly-lysine and carboxymethyl cellulose, generating the overall complex referred to as “poly-ICLC” (polyinosinic poly-cytidylic acid). Methods of preparation and clinical use of poly-ICLC was initially described in U.S. Pat. No. 4,349,538 (Levy), incorporated herein by reference, and further described in Worldwide Patent WO2005102278A1 (Salazar).

Administration of Poly-ICLC results in multiple clinical actions including interferon induction, broad immune enhancements, and regulation or activation of various genes and enzymes. Due to these effects, poly-ICLC has been broadly considered as an antitumor agent, an antiviral, or an adjuvant.

The size of the poly-I and poly-C components of poly-ICLC (or previously, poly-IC) has been correlated to efficiency of interferon induction (Levy 1981). More recently the mechanism of this differential effect has been further elucidated. For example, short poly-IC preferentially activates the RIG-1 helicase with certain antiviral effects, while long chain poly-IC activates the MDAS helicase, resulting in a broader immunomodulation, adjuvant and antiinflammatory action. Nevertheless because of the complex and inter-related clinical actions of Poly-ICLC, the overall correlation between polyribonucleotide component size and each facet of biological activity is not fully understood. Despite lacking complete understanding, it is clear that precise control of the size of the polyribonucleotide components is a highly desirable element of any production method. Targeting specific molecular weight ranges may enhance activity toward a certain indication or reduce toxicity at effective dose levels. To date, however, a reliable method of producing Poly-ICLC with a desired range of molecular weights has been unavailable.

### SUMMARY OF THE INVENTION

The present invention improves biocatalytic polyribonucleotide production by immobilization of the PNPase biocatalyst, by providing an economic scalable process and by controlling the range of molecular weights of the polyribonucleotide product by controlling certain elements of the process.

The invention includes polynucleotide phosphorylase which has been covalently attached to an amino-functionalized solid support via a glutaraldehyde linkage, a scalable process based on repeatedly reacting inosine diphosphate or cytidine diphosphate monomers with immobilized polynucleotide phosphorylase to produce polyribonucleotide chains, and a process for controlling the range of molecular weights of polyribonucleotide chains by varying the concentration of certain input components and by varying the reaction time of the process.

A scalable process for production of polyribonucleotides of controlled molecular weight range through variation of processing time and input concentrations. Key elements include a method for immobilization of polynucleotide phosphorylase which has been covalently attached to an amino-functionalized solid support via a glutaraldehyde linkage; a method of repeatedly reacting inosine diphosphate or cytidine diphosphate monomers with immobilized polynucleotide phosphorylase to produce polyribonucleotide chains; control of the chain length of Poly(I) and Poly(C) by varying cofactor concentration and the length of reaction time; a method for controlled and efficient large-scale manufacture of a specific, determined range of molecular weight poly I and poly C homopolymer chains.

### BRIEF DESCRIPTION OF THE DRAWINGS

FIG. 1 is a flow chart illustrating an overview of the basic process.

FIG. 2 is a schematic for determining appropriate cofactor concentration to generate a specific size of polymer in a given time.

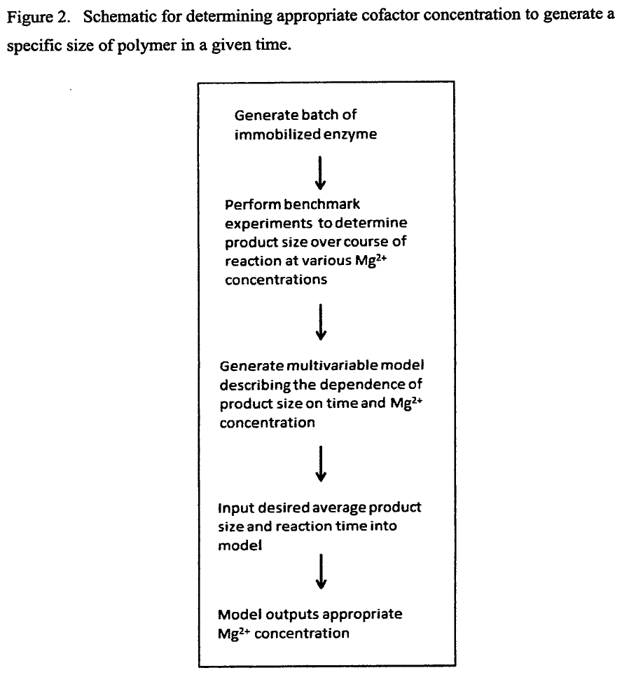

FIG. 3 shows the results of an experiment testing Immobilized Enzyme Activity Over Repeated Cycles of Poly-I Production as measured by depletion of substrate monomer.

FIG. 4 shows the results of an experiment testing Immobilized Enzyme Activity Over Repeated Cycles of Poly-C Production as measured by depletion of substrate monomer.

FIG. 5 shows the results of an experiment demonstrating control of polymer size by varying reaction duration.

FIG. 6 shows the results of an experiment demonstrating control of Poly-C polymer size by varying magnesium cofactor concentration.

FIG. 7 shows various size poly I and poly C preparations used in the confirmatory experiments.

FIG. 8 shows the dose titration curves IFN-I production responses of various poly-ICLC preparations made with different molecular weight poly-I and poly-C homopolymers.

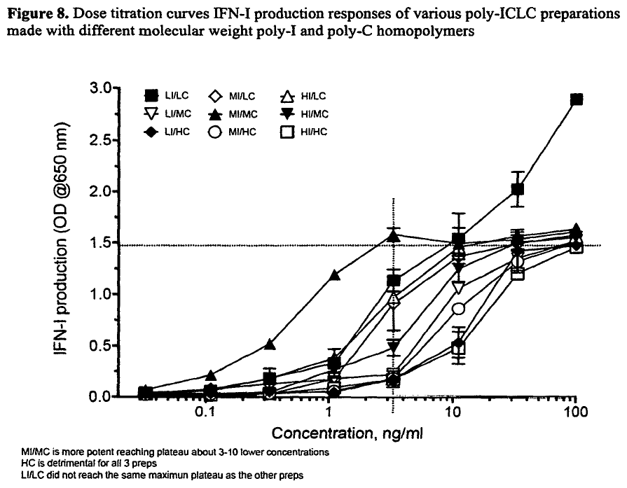

FIG. 9 shows the IFN-I production by a reporter cell line induced by various preparations of poly-ICLC at 3.3 ng/ml made with different molecular weight poly-I and poly-C homopolymers.

### DESCRIPTION OF THE PREFERRED EMBODIMENT

The present invention utilizes PNPase, which may be any source, but is preferably from a recombinant source and free of protease, nuclease, and phosphatase and most preferably is of bacterial origin, especially from E. coli or B. stearothermophylus.

The solid support comprises a methacrylate resin with pore diameters from 300-1800 Å and functionalized with an amino group. Most preferably, pore diameter is from 1200-1800 Å and the amino group is attached with a short spacer. Though any crosslinking agent may be used, glutaraldehyde is preferred to create an imine linkage. The present invention has found most improved PNPase stability and least reduced PNPase activity with a methacrylate support and glutaraldehyde-mediated amino linkage.

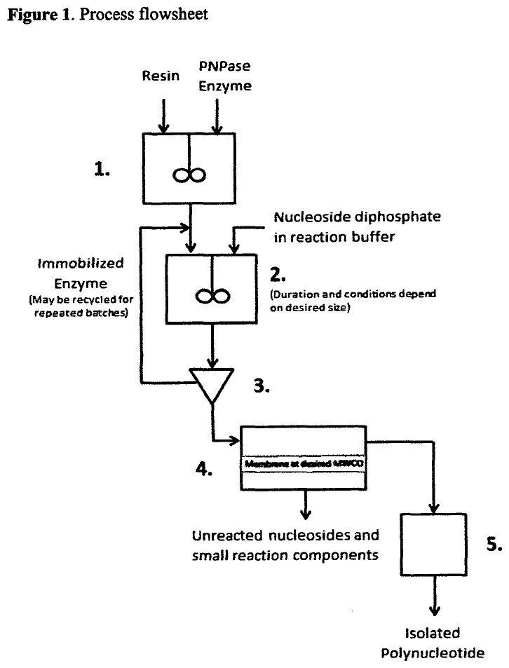

Referring to FIG. 1, in overview the process comprises five steps.

### Step 1—Enzyme Immobilization

Immobilization is performed by contacting an aqueous enzyme with previously activated methacrylate amino resin. The aqueous solution is typically buffered at low concentration (0.01-0.05 M) and the ratio of resin to aqueous enzyme ranges from 1 to 1 (w/v) to 1 to 20 (w/v), but is most preferably 1 to 4 (w/v). Contact time between the enzyme and support is typically 18 hours at 25° C. with gentle mixing, but may range from 12 to 36 hours. Any unbound PNPase is subsequently removed by filtration, although immobilization efficiency tends to be very high under these conditions.

### Step 2—Batch Biocatalysis

Examples of nucleoside diphosphates to be polymerized include inosine diphosphate (IDP) and cytidine diphosphate (CDP), but may also include any natural or synthetic nucleoside diphosphates.

Polyribonucleotide production is performed by contacting an aqueous solution of nucleoside diphosphate with immobilized PNPase. The aqueous solution consists of a buffer, cofactor, reducing agent, metal chelator, and the nucleoside diphosphates. The buffer is most preferably tris at a pH between 7 and 9. The cofactor is most preferably Mg²⁺ at concentrations between 2 and 50 mM. The reducing agent is most preferably Tris(2-carboxyethyl) Phosphine (“TCEP”) at concentrations between 0.1 and 5 mM. The metal chelator is most preferably Ethylene diaminetetraacetic acid (“EDTA”) at concentrations between 0.1 and 5 mM. Nucleoside diphosphates may be any of the previously described monomers at concentrations from 1-10 g/L. Ratio of the immobilized enzyme to aqueous solution ranges from 1 to 1 (w/v) to 1 to 50 (w/v) and is most preferably 1 to 20 (w/v).

Once contacted, the reaction is typically incubated at elevated temperature with gentle mixing. Temperatures range from 30° C. to 50° C. and are most preferably 37° C. Reaction time can be varied from 16 h to 72 h. The reaction typically approaches maximum yield of polynucleotide products by 20 h. Extending reaction time beyond this point has minimal effect on yield but results in a decrease in the average size of polyribonucleotide products.

### Step 3—Coarse Filtration

After reaction, the supernatant is filtered off the immobilized enzyme resin by vacuum and the resin is washed with an equal volume of buffered aqueous solution. Washed resin is suitable for repeated reaction cycles, displaying retention of >95% activity after 6 cycles. The polynucleotide products are contained in the supernatant and resin wash

### Step 4—Tangential Flow Filtration

The polynucleotide products are then isolated from smaller buffer components by tangential flow filtration. Because the difference in size of the polynucleotide products and smaller impurities spans several orders of magnitude, the acceptable molecular weight cutoff (MWCO) of the membrane ranges from 1,000 to 100,000 Da. The most preferable MWCO depends on the exact size of the polynucleotide produced in a given reaction, however a size of 10,000 Da is suitable for most applications. Acceptable types of membrane modules include spiral wound and hollow fiber. During tangential flow filtration, the large polynucleotide products are retained on the feed side of the membrane, while smaller impurities pass through into the permeate. Impurity-free water is continuously added to the feed side, matching the rate of permeation. Addition of approximately 20 times the sample volume of water is required to fully eliminate smaller impurities.

### Step 5—Lyophilization

The retained material from the tangential flow filtration step—isolated polynucleotide in water—is suitable for lyophilization to produce a solid product.

The process has several advantages over the prior art.

Choay (FR 1970), Rokugawa (JP 1972), and De Lassauniere (FR 1988) each disclose a method of homogeneous biocatalytic production of polyribonucleotide, utilizing soluble PNPase enzyme to produce soluble products. The PNPase must be removed during product isolation, which is commonly achieved by extraction with phenol, chloroform octanol (Choay), or ethanol and other nondisclosed solvents (Rokugawa). In each case, this extraction is destructive and precludes reuse of enzyme in subsequent batches. This aspect limits the scalability of these processes due to the high price of enzyme. The present invention describes enzyme immobilization on a solid support. In addition to simplifying enzyme removal (by coarse filtration), this heterogeneous biocatalysis approach is nondestructive and allows enzyme reuse. PNPase immobilized on solid support has been demonstrated to retain activity over several reaction cycles, allowing efficient use of enzyme at scale.

While Kise (JP1988) reports a 40% coupling efficiency (of PNPase to support) using chitosan, the present invention achieves coupling efficiencies in excess of 98% using methacrylate.

Choay (FR 1973) couple PNPase to activated sepharose, using cyanogen bromide as an activating agent. This reagent is acutely toxic and its use is preferably avoided in a drug product for human use.

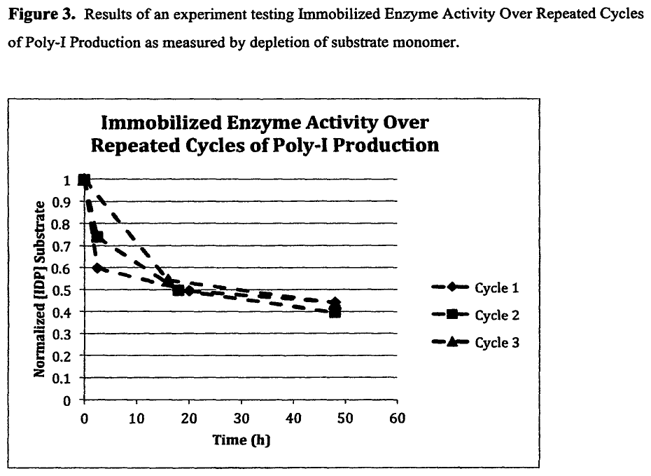

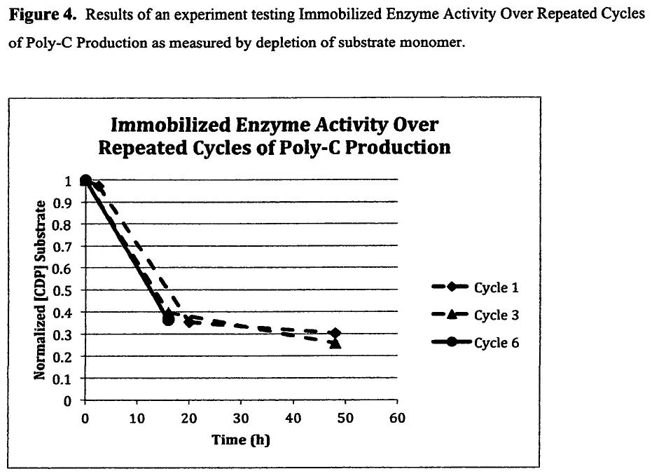

Note further that the process does not involve the use of molecular oxygen (which may require sparging) or require controlled pH (which may require pumps and large quantities of acid or base). Furthermore, the immobilized biocatalyst is reusable. On the solid phase support, PNPase is easily separated from the aqueous phase post-reaction and, as shown in the experimental results shown in FIGS. 3 and 4, does not show loss of activity over at least six reaction cycles. This reusability allows for multiple reaction cycles and the production of at least an order of magnitude more polynucleotide from a given amount of enzyme and reactor size. Therefore the process is scalable.

Immobilization has two main components: the identity of the (typically polymer) backbone and the linkage length and chemistry (for attaching enzyme). While Moran 1989 reports various acrylic supports but epoxy linkage chemistry, and Kise 1989 reports imide linkage chemistry but on a chitosan support, neither reports or suggests the imine (glutaraldehyde-mediated) linkage chemistry with a C2 spacer and methacrylate backbone of the current invention.

It has been discovered that both reaction duration and cofactor concentration modulate product size. Generally, a longer the duration of reaction results in a lower the average molecular weight of polynucleotide products. This effect has been noted in previous patent literature. (Moran (US 1989) and Kise (JP 1989)). We have discovered that higher concentrations of Mg²⁺ in the reaction will also result in a lower average molecular weight of polynucleotide products. Using a preliminary set of benchmark experiments, a multivariable model describing the dependence of polynucleotide size on time and Mg²⁺ concentration is readily established for a batch of immobilized enzyme. By inputting an arbitrary time in this model, a Mg²⁺ concentration can be selected that will produce product of a desired size range in that given time.

De Lassauniere 1988 observed that adding a high concentration of Mg²⁺ upfront results in a different final product size than adding smaller amounts over the course of the reaction, but does not teach that varying upfront concentration will result in variously-sized polymers or how to use initial concentration to produce a specific, desired range of sizes.

Moran (US 1989) and Kise (JP 1989) each identify reaction duration as a determinant of final polyribonucleotide size, however both fail to identify cofactor [Mg²⁺] as an additional modulator of polymer size. Relying solely on reaction duration to determine polyribonucleotide size has several disadvantages, such as very long reaction durations if small products are desired and variable reaction durations when producing various product sizes.

Notably, the present invention describes the dual levers of co-factor concentration and reaction length to modulate polymer product size range. This allows the synthesis of product of desired size within a specific timeframe and has notable advantages over previous processes that rely solely on reaction length, such as the flexibility to fit the process into specific manufacturing windows or shift schedules. The multifactor approach of the current invention allows rapid generation of even small products, the ability to tailor reaction duration to manufacturing shift schedules, and the ability to produce variably-sized products in multiple batches all using the same process duration.

### EXAMPLES

### Example 1. PNPase Immobilization on Pre-Activated Amino Methacrylate Beads

Purolite resin (ECR8315) was washed with 2 mL immobilization buffer (50 mM Tris, pH 8.5, 2 mM TCEP, and 1 mM EDTA) and filtered. Resin was activated by addition of 8 mL of immobilization buffer containing 2% glutaraldehyde. After 60 minutes of incubation at 20° C., the beads were filtered and washed with an additional 8 mL of immobilization buffer. 8 KU PNPase (Nipro) was dissolved in 8 mL immobilization buffer. To initiate immobilization, the PNPase solution was added to two grams of activated resin (wet weight). The slurry was mixed gently for 18 h at 25° C. The liquid phase was filtered, collected, and assayed, indicating an immobilization efficiency of >98%, confirming that PNPase can efficiently be attached to methacrylate beads via imide chemistry. The resin was washed twice with 8 mL immobilization buffer. Immobilized PNPase resin was stored at 4° C.

### Example 2. Repeated Production of Poly-I and Poly-C with Immobilized PNPase

To demonstrate that the process could be scaled up by repetition, an experiment was carried out to measure the preservation of the substrate over repeated cycles. Enzyme activity is typically understood as the ability of an enzyme to convert a certain amount of substrate in a given time. Conversion is typically measured by evolution of product, but can be measured equivalently by consumption of substrate, the method chosen here.

Inosine diphosphate (IDP) was dissolved to a final concentration of 10 g/L in reaction buffer (50 mM tris, pH 9.0, 5 mM MgCl₂, 20 mM KCl, 1 mM TCEP, 1 mM EDTA). One mL of 10 g/L IDP solution was added to 50 mg of previously prepared immobilized PNPase resin. The slurry was gently agitated on a rotary tube rotator at 37° C. for 48 hours. At several times over the course of reaction, resin was allowed to settle and supernatant was sampled. Samples were run on HPLC to determine remaining concentration of IDP in solution. After 48 hours, resin was coarsely filtered using vacuum and washed with an equal volume of reaction buffer lacking substrate. Resin was transferred to a fresh reaction vessel and a fresh solution of 10 g/L IDP was added. A second 48 hour reaction was performed as previously described. This overall scheme was repeated for 3 cycles with the results shown in FIG. 3. As FIG. 3 shows, the consumption of substrate (IDP, in this case) over time for 3 repeated reaction cycles, which, as just described, is related to the activity at each cycle. The curves generally overlay and all reach ˜60% conversion in 48 h (that is, 40% substrate remaining, or 0.4 when normalized), showing that the immobilized enzyme maintains the same activity across the repeated reactions.

The immobilized enzyme appeared to lose no activity over 3 reaction cycles at elevated temperature. An extended set of experiments indicated that >95% activity is retained over an additional 3 cycles. Notably, the 6 total cycles were run over a time period of 1.5 months (with intermittent storage at 4° C. between cycles), indicating that immobilization holistically mitigates the destabilizing effects of enzymatic turnover, temperature, and time.

Similar experiments were conducted using a different substrate, 10 g/L cytidine diphosphate (CDP) solution in place of the 10 g/L IDP solution (FIG. 4). These experiments were extended to additional reaction cycles and retention of >95% activity was observed over six cycles. (FIG. 4 shows only the first and last cycles and one intermediate cycle; some intermediate cycle data is omitted to enhance readability of the figure.) Notably, rigorous washing of the resin was required for activity between cycles, presumably to dissociate polymerized product from the immobilized enzyme.

Thus, PNPase shows polymerase activity using IDP or CDP substrates when immobilized on methacrylate beads via imide chemistry. Further, this activity does not measurably decrease over several reaction cycles. Because activity is not lost, the same immobilized enzyme can be used over multiple reaction cycles, confirming that the reaction is scalable.

### Example 3. Modulation of Polynucleotide Size by Varying Reaction Length

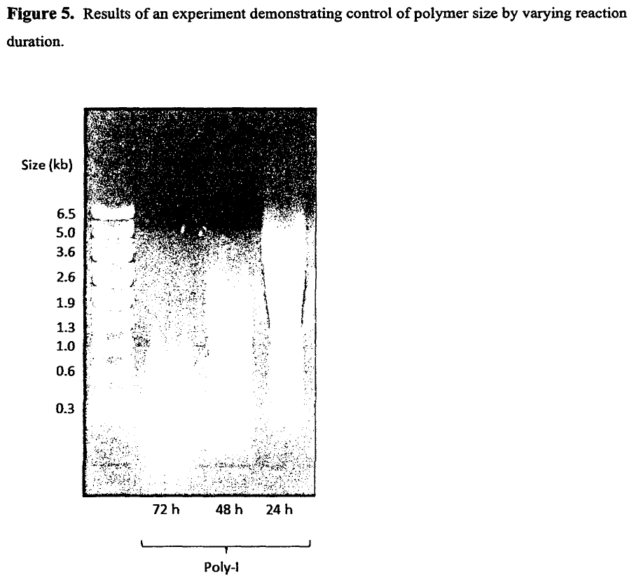

Inosine diphosphate (IDP) was dissolved to a final concentration of 10 g/L in modified reaction buffer (50 mM tris, pH 8.5, 5 mM MgCl₂, 1 mM TCEP, 1 mM EDTA). One mL of 10 g/L IDP solution was added to 50 mg of previously prepared immobilized PNPase resin. The slurry was gently agitated on a rotary tube rotator at 37° C. for 72 hours. At 24, 48, and 72 hours, resin was allowed to settle and reaction supernatant was sampled and immediately frozen. At the culmination of the experiment, samples were thawed and resolved by agarose gel electrophoresis. Each sample produced a smeared band on the agarose gel, indicative of polydispersed products (FIG. 5). The 24 h reaction produced material mainly in the 1.5-4 kb range. The 48 h reaction produced material mainly in the 0.5-1.3 kb range. The 72 h reaction produced material mainly in the <0.3-0.6 kb range. Therefore, polynucleotide product size can be modulated by variations in reaction length.

### Example 4. Modulation of Poly-I Size by Varying Cofactor Concentration

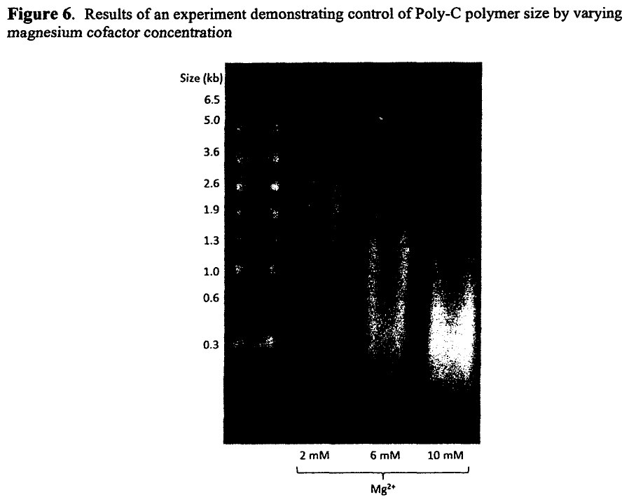

Inosine diphosphate (IDP) was dissolved to a final concentration of 10 g/L in modified reaction buffers, all containing 50 mM tris, pH 8.5, 1 mM TCEP, 1 mM EDTA, but varying MgCl₂ concentration at 2 mM or 10 mM. One mL of 10 g/L IDP solution at each MgCl₂ concentration was added to 50 mg of previously prepared immobilized PNPase resin. Each slurry was gently agitated on a rotary tube rotator at 37° C. for 48 hours. At 48 hours, resin was allowed to settle and each reaction supernatant was sampled and resolved by agarose gel electrophoresis. Each sample produced a smeared band on the agarose gel, indicative of polydispersed products (FIG. 6). The size of the products varied based on MgCl₂ concentration, with the 2 mM reaction producing material mainly in the 1.5-5 kb range and the 10 mM reaction producing material mainly in the <0.3-0.6 kb range.

A polynucleotide product centered at 1 kb was desired in a reaction of 48 hour duration. The previously described data was interpolated according to the multivariable model relating size, duration, and Mg²⁺ concentration, resulting in a recommended Mg²⁺ concentration of 6 mM. Inosine diphosphate (IDP) was dissolved to a final concentration of 10 g/L in modified reaction buffer, containing 50 mM tris, pH 8.5, 1 mM TCEP, 1 mM EDTA, and 6 mM Mg²⁺. One mL of 10 g/L IDP solution was added to 50 mg of previously prepared immobilized PNPase resin. The slurry was gently agitated on a rotary tube rotator at 37° C. for 48 hours. At 48 hours, resin was allowed to settle and the reaction supernatant was sampled and resolved by agarose gel electrophoresis. The sample produced a smeared band on the agarose gel, indicative of polydispersed products (FIG. 6). As predicted, the 6 mM reaction produced material mainly in the 0.5-1.5 kb range. Therefore, Poly-I of a desired size can be produced in a given timeframe by varying the concentration of cofactor present in the reaction.

### Example 5. Modulation of Poly-C Size by Varying Cofactor Concentration

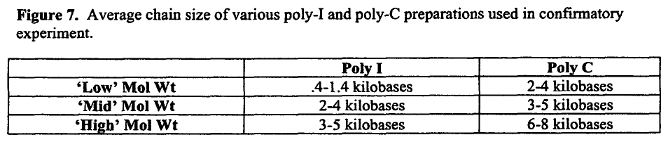

Cytidine diphosphate (CDP) was dissolved to a final concentration of 10 g/L in modified reaction buffers, all containing 50 mM tris, pH 8.5, 1 mM TCEP, 1 mM EDTA, but varying MgCl₂ concentration at 5 mM or 25 mM. One mL of 10 g/L IDP solution at each MgCl₂ concentration was added to 50 mg of previously prepared immobilized PNPase resin. Each slurry was gently agitated on a rotary tube rotator at 37° C. for 48 hours. At 48 hours, resin was allowed to settle and each reaction supernatant was sampled and resolved by agarose gel electrophoresis. Each sample produced a smeared band on the agarose gel, indicative of polydispersed products (FIG. 7). The size of the products varied based on MgCl₂ concentration, with the 5 mM reaction producing material mainly in the 5-6.5+kb range and the 25 mM reaction producing material mainly in the 0.5-2 kb range.

A polynucleotide product centered at 3 kb was desired in a reaction of 48 hour duration. The previously described data was interpolated according to the multivariable model relating size, duration, and Mg²⁺ concentration, resulting in a recommended Mg²⁺ concentration of 25 mM. Cytidine diphosphate (CDP) was dissolved to a final concentration of 10 g/L in modified reaction buffer, containing 50 mM tris, pH 8.5, 1 mM TCEP, 1 mM EDTA, and 25 mM Mg²⁺. One mL of 10 g/L CDP solution was added to 50 mg of previously prepared immobilized PNPase resin. The slurry was gently agitated on a rotary tube rotator at 37° C. for 48 hours. At 48 hours, resin was allowed to settle and the reaction supernatant was sampled and resolved by agarose gel electrophoresis. The sample produced a smeared band on the agarose gel, indicative of polydispersed products (FIG. 7). As predicted, the 15 mM reaction produced material mainly in the 2-4 kb range. Therefore, Poly-C of a desired size can be produced in a given timeframe by varying the concentration of cofactor present in the reaction.

### Experimental Biological Activity Confirmation

The present invention has measurable effect on the biological activity of pharmaceuticals manufactured using the specified poly-I and poly-C sizes. Specifically, poly-ICLC was produced using low, middle and high molecular weight preparations of poly-I and poly-C that had been manufactured as described above in the Claims and the Preferred embodiment. The average sizes in kilobases (kb) for each are shown in FIG. 1.

Nine preparations of poly-IC were made by mixing all possible combinations:

$$1. LMW poly - I + LMW poly - C = LI / LC$$

$$2. LMW poly - I + MMW poly - C = LI / MC$$

$$3. LMW poly - I + HMW poly - C = LI / HC$$

$$4. MMW poly - I + LMW poly - C = MI / LC$$

$$5. MMW poly - I + MMW poly - C = MI / MC$$

$$6. MMW poly - I + HMW poly - C = MI / HC$$

$$7. HMW poly - I + LMW poly - C = HI / LC$$

$$8. HMW poly - I + MMW poly - C = HI / MC$$

$$9. HMW poly - I + HMW poly - C = HI / HC$$

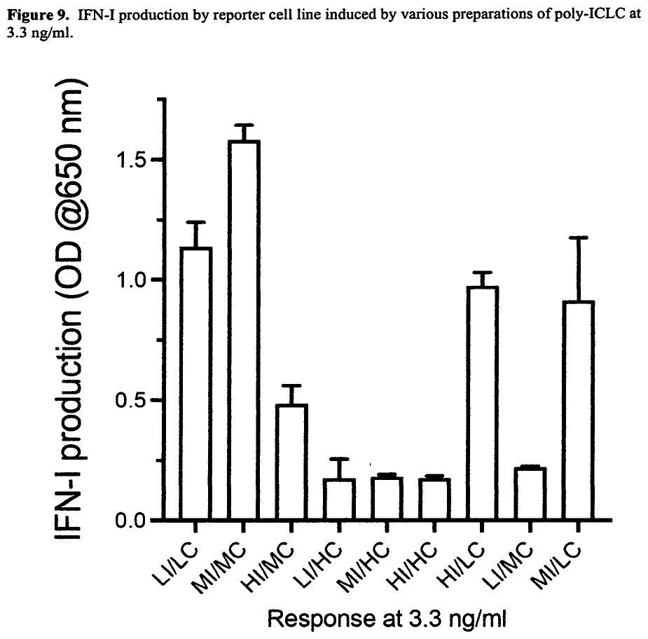

After preparing the poly-I/poly-C duplexes, these were combined with carboxymethyl cellulose (CMC) and poly-Lysine to make 9 different preparations of poly-ICLC. The 9 poly-ICLC preparations were tested at various concentrations for their ability to induce IFN-I production using a human reporter cell line. Results of the dose titration curves are shown in FIG. 9. Comparison of responses using these preparations at a concentration of 3.3. ng/ml is shown in FIG. 10.

The results indicate an unexpectedly large difference in the biological activity among the various preparations. For example: A) The MI/MC combination exhibited the highest potency as compared to the other preparations. B) Surprisingly, all 3 preparations made with HC, which is the largest polynucleotide, had lower activity as compared to the rest. C) The 3 preparations made with LC had similar activity.

While specific embodiments of the invention have been shown and described in detail to illustrate the application of the principles of the invention, it will be understood that the invention may be embodied otherwise without departing from such principles and that various modifications, alternate constructions, and equivalents will occur to those skilled in the art given the benefit of this disclosure.

What is claimed is:

1. A process for immobilization of polynucleotide phosphorylase comprising:
providing an amino-functionalized solid support; and covalently attaching said polynucleotide phosphorylase to said amino-functionalized solid support via a glutaraldehyde linkage.

2. The process of claim 1 wherein the solid support comprises a methacrylate resin with pore diameters from 300-1800 Å and functionalized with an amino group.

3. The process of claim 2 wherein said pore diameter is from 1200-1800 Å and the amino group is attached with a short spacer.

4. A scalable process for producing polyribonucleotide chains, comprising:
repeatedly reacting inosine diphosphate or cytidine diphosphate monomers with immobilized polynucleotide phosphorylase so as to produce polyribonucleotide chains by:
conducting an initial contact phase consisting of contacting an aqueous enzyme with an activated methacrylate amino resin so as to create immobilized PNPase;
conducting a second contact phase consisting of contacting an aqueous solution, comprising a buffer, a cofactor, a reducing agent, a metal chelator and nucleoside diphosphate with said immobilized PNPase so as to create a polyribonucleotide solution;
conducting a filtering phase consisting of filtering the solution into a filtrate and a supernatant so as to remove immobilized enzyme resin into the filtrate, leaving polyribonucleotides in the supernatant;
recovering the immobilized enzyme resin;
repeating said second contact phase, introducing the recovered immobilized enzyme resin in said second contact phase;
repeating said filtering phase.

5. The process of claim 4 wherein said buffer is tris at a pH between approximately 7 and 9.

6. The process of claim 4 wherein said cofactor is a divalent metal cation.

7. The process of claim 6 wherein said divalent metal cation is Mg²⁺ at concentrations between approximately 2 and 50 mM.

8. The process of claim 4 wherein said reducing agent is TCEP at concentrations between 0.1 and 5 mM.

9. The process of claim 4 wherein said metal chelator is EDTA at a concentration between approximately 0.1 and 5 mM.

10. A process for producing polyribonucleotide chains having a predetermined range of molecular weights, comprising:
conducting an initial contact phase consisting of contacting an aqueous enzyme with an activated methacrylate amino resin so as to create immobilized PNPase;
contacting, for a predetermined incubation time, an aqueous solution, comprising a buffer, a cofactor at a predetermined concentration, a reducing agent, a metal chelator and nucleoside diphosphate with said immobilized PNPase so as to create a polyribonucleotide;
conducting a filtering phase consisting of filtering the solution into a filtrate and a supernatant so as to remove immobilized enzyme resin into the filtrate, leaving polyribonucleotides in the supernatant;
further filtering the supernatant using tangential flow filtration so as to separate the polyribonucleotides from smaller buffer components and other impurities.

11. The process of claim 10 wherein said predetermined incubation time and predetermined concentration of said cofactor is determined by in-process testing.

12. The process of claim 10 further comprising:
recovering the immobilized enzyme resin produced in and repeating the process of claim 10 using said recovered immobilized enzyme.

13. The process of claim 10 wherein said nucleoside diphosphate is either inosine diphosphate or cytidine diphosphate.

14. The process of claim 10 wherein said buffer is tris at a pH between approximately 7 and 9.

15. The process of claim 10 wherein said cofactor is a divalent metal cation.

16. The process of claim 15 wherein said divalent metal cation is Mg²⁺ at concentrations between approximately 2 and 50 mM.

17. The process of claim 10 wherein said reducing agent is TCEP at concentrations between 0.1 and 5 mM.

18. The process of claim 10 wherein said metal chelator is EDTA at a concentration between approximately 0.1 and 5 mM.

19. A process for producing Poly-I in approximately the 0.3-0.6 kb range, comprising:
dissolving inosine diphosphate to a final concentration of 10 g/L in a reaction buffer comprising 50 mM tris, pH 8.5, 1 mM TCEP, 1 mM EDTA, 10 mM MgCl₂ so as to create an IDP solution; and
adding 1 mL of said IDP solution to 50 mg of immobilized PNPase resin and gently agitating at approximately 37° C. for approximately 48 hours.

20. A process for producing Poly-I in approximately the 0.5-2 kb range, comprising:
dissolving inosine diphosphate to a final concentration of 10 g/L in a reaction buffer comprising 50 mM tris, pH 8.5, 1 mM TCEP, 1 mM EDTA, 25 mM MgCl₂ so as to create an IDP solution; and
adding 1 mL of said IDP solution to 50 mg of immobilized PNPase resin and gently agitating at approximately 37° C. for approximately 48 hours.

21. A process for producing Poly-I in approximately the 1.5-5 kb range, comprising:
dissolving inosine diphosphate to a final concentration of 10 g/L in a reaction buffer comprising 50 mM tris, pH 8.5, 1 mM TCEP, 1 mM EDTA, 2 mM MgCl₂ so as to create an IDP solution; and
adding 1 mL of said IDP solution to 50 mg of immobilized PNPase resin and gently agitating at approximately 37° C. for approximately 48 hours.

22. A process for producing Poly-I in approximately the 5-6.5 kb range, comprising:
dissolving inosine diphosphate to a final concentration of 10 g/L in a reaction buffer comprising 50 mM tris, pH 8.5, 1 mM TCEP, 1 mM EDTA, 5 mM MgCl₂ so as to create an IDP solution; and
adding 1 mL of said IDP solution to 50 mg of immobilized PNPase resin and gently agitating at approximately 37° C. for approximately 48 hours.

23. A process for producing Poly-C in approximately the 0.5-2.0 kb range, comprising:
dissolving cytidine diphosphate to a final concentration of 10 g/L in a reaction buffer comprising 50 mM tris, pH 8.5, 1 mM TCEP, 1 mM EDTA, 25 mM MgCl₂ so as to create an IDP solution;
adding 1 mL of said IDP solution to 50 mg of immobilized PNPase resin and gently agitating at approximately 37° C. for approximately 48 hours.

24. A process for producing Poly-C in approximately the 5-6.5 kb range, comprising:
dissolving cytidine diphosphate to a final concentration of 10 g/L in a reaction buffer comprising 50 mM tris, pH 8.5, 1 mM TCEP, 1 mM EDTA, 5 mM MgCl₂ so as to create an IDP solution;
adding 1 mL of said IDP solution to 50 mg of immobilized PNPase resin and gently agitating at approximately 37° C. for approximately 48 hours.

25. A process for producing Poly-C in approximately the 2-4 kb range, comprising:
dissolving cytidine diphosphate to a final concentration of 10 g/L in a reaction buffer comprising 50 mM tris, pH 8.5, 1 mM TCEP, 1 mM EDTA, 15 mM MgCl₂ so as to create an IDP solution;
adding 1 mL of said IDP solution to 50 mg of immobilized PNPase resin and gently agitating at approximately 37° C. for approximately 48 hours.
